(19) United States
(12) Patent Application Publication
(10) Pub. No.: US 2018/0303669 A1
(43) Pub. Date: Oct. 25, 2018

(54) BILATERAL COMPRESSION DEVICE

(71) Applicant: John McCann, Sandy, UT (US)

(72) Inventor: John McCann, Sandy, UT (US)

(21) Appl. No.: 15/496,830

(22) Filed: Apr. 25, 2017

Related U.S. Application Data
(63) Continuation of application No. 15/491,583, filed on Apr. 19, 2017, now abandoned.

(51) Int. Cl. A61F 9/02; A61F 9/04; A61F 13/12; A61H 1/00

(52) U.S. Cl. CPC A61F 9/026; A61F 9/04; A61H 2205/025; A61H 1/008; A61H 2205/022; A61F 13/124

(57) ABSTRACT

The present invention provides an improved bilateral compression device for post-operative surgical site, the bilateral compression device including a central cavity presented by an outerwall and a circumscribing sidewall, the central cavity in receipt of a post-operative pillow further comprising an outer membrane separated from an inner membrane for exerting a central compressive force towards the post-operative surgical area which varies from a surrounding compression force.

### FIELD OF THE INVENTION

The present invention is broadly directed to post operative treatment devices and, more particularly, to an improved post-operative bilateral compression device with contoured surfaces to reduce pain, diminish swelling and avoid fluid accumulation after ophthalmic procedures.

### BACKGROUND OF THE INVENTION

Ophthalmological procedures general provide the most significant improvement in the quality of life. It is estimated that cosmetic eyelid surgery is the third most popular elective cosmetic procedure in the United States and it is anticipated that ophthalmology will experience the greatest growth in demand in the coming years based on an aging population and increased treatment success rates.

As a result of the increased number of surgical procedures, many people will experience the typical post-operative problems of surgery like swelling, pain and bruising which contribute to an extended healing period due to accumulation of fluids commonly referred to as seroma. Patients frequently complain about the bruising of the eyelids and the area surrounding the post-operative surgical area. Bruising of the eyelids may contribute to poor lid globe apposition which may lead to blurred vision and cause additional delays in the patient recovery.

In typical post-operative procedures, it is recommended to utilize the PRICE principals, namely to protect, rest, ice, compress and elevate around the affected areas to help them heal. However, the facial anatomy associated with typical ophthalmic surgical procedures presents unique difficulties which are effected by surrounding tissue, cartilage and bones which interfere with the PRICE techniques. In addition, maintaining compression and ice on the ophthalmic post-operative areas can create physical and psychological discomforts upon the treated patient.

For many years eyelid surgeons have recommended frequent ice compresses for the first few days after surgery to minimize bruising. Ice and cooling therapies generally help decrease blood loss, bruising, swelling, pain sensation and a general increase in the rate of recovery. However, the eyelids have a robust vascular supply and only a thin layer of skin to conceal bruising so that substantial bruising still occurs with heated and cooling therapies. In addition, the eye area is subject to swelling and “puffiness” which is part of a patients genetic predisposition, allergies, the aging process, lack of sleep, dietary considerations, and lifestyle considerations.

Because of the large vascular network around the eye, the eye area is subject to swelling which can increase during times of allergies, headaches, fatigue, edema, hangovers, environmental conditions, and medical and non-medical treatments including massages, botox, and surgeries. It would therefore be beneficial to have a device for use during the post-operative process which helps reduce bruising of the eyelids and area surrounding the post-operative surgical area.

In the past, one solution has been to provide eye patches to help with recovery during the post-operative process around the post-operative surgical area. However, eye patches are not acceptable for cases when both eyes are undergoing recovery for an extended period of time because they can create additional anxiety issues and physical limitations for the patient during the recovery process.

Accordingly, there is a need for an improved post operative ophthalmologic device which is removable while providing bilateral compression during use to assist the patient during the recovery period and addresses at least a portion of the aforementioned shortcomings.

### SUMMARY OF THE INVENTION

The present invention includes an improved bilateral compression device for post-operative surgical site, the bilateral compression device including a central cavity presented by an outerwall 18 and a circumscribing sidewall 12, said central cavity in receipt of a post-operative pillow further comprising an outer membrane separated from an inner membrane for exerting a central compressive force towards the post-operative surgical area which varies from a surrounding compression force.

Various objects and advantages of the present invention will become apparent from the following description taken in conjunction with the accompanying drawings wherein are set forth, by way of illustration and example, certain embodiments of this invention. The drawings submitted herewith constitute a part of this specification, include exemplary embodiments of the present invention, and illustrate various objects and features thereof.

### BRIEF DESCRIPTION OF THE DRAWINGS

FIG. 1 is a left side perspective view of an exemplary embodiment of the present invention.

FIG. 2 is an exploded side perspective of the exemplary embodiment of FIG. 1.

FIG. 3 is a rear perspective of the exemplary embodiment of FIG. 1.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Referring to the drawings in more detail, the reference numeral 10 generally refers to an embodiment of the present invention, a bilateral compression device designed to be worn on the head of the patient after eye surgery to assist in the healing process. The bilateral compression device 10 includes a central cavity 20 presented by an outerwall 18 and a circumscribing sidewall 12 which generally extends along the outerwall 18.

Example materials which may be used for the outerwall 18 and circumscribing sidewall 12 include but are not limited to plastics and metals. In one embodiment, the outerwall 18 and circumscribing sidewall 12 may comprise a single rigid piece for example using an injection molding process. In one exemplary embodiment, the bilateral compression device 10 may utilize a google like one commercially available from Smith Optics, Inc. However, in an alternative embodiment, the outerwall 18 and circumscribing sidewall 12 may be flexible or fabricated as multiple pieces that can be selectively assembled and disassembled as desired. In yet another exemplary embodiment, the outerwall 19 and circumscribing sidewall 12 may be made from different materials, each having different properties and different shapes as desired.

The circumscribing sidewall 12 generally extends from the outerwall 18 providing a rim and presenting a pair of slotted openings 14 which generally receive an elastic band 16 for easy removal and adjustment of the bilateral compression device 10. The elastic band 16 also allows the bilateral compression device 10 to remain on during the night while the patent sleeps. Generally, the elastic band 16 is provided that generally includes an elastic or resilient material to allow stretching of the elastic band 16 as desired for placement or removal of the bilateral compression device 10. Generally, the elastic band 16 with the outerwall 18 and circumscribing sidewall 12 presents a biased contoured surface for applying an even gentle pressure to the eyelids and the periocular region which is referred to generally as the post operative surgical site which includes the facial contours associated with the eye socket and the nasal bone (not shown).

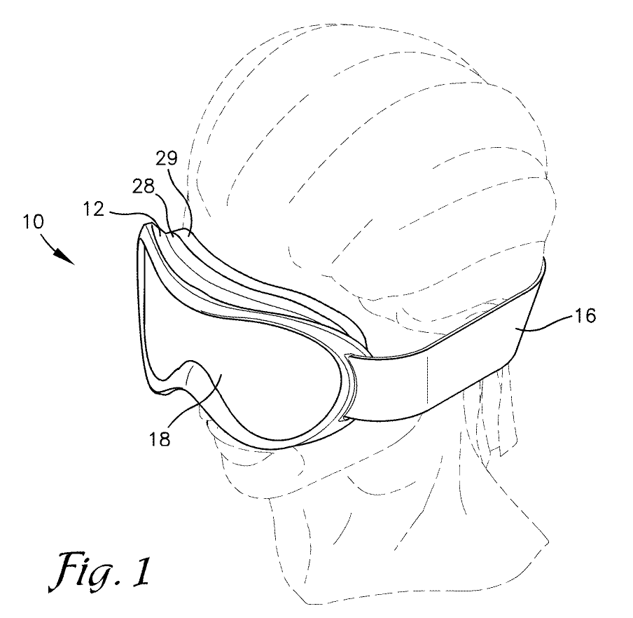

The bilateral compression device 10 generally protects the post-operative surgical area with the circumscribing sidewall 12 extending along the outer area of the post-operative surgical area. In the depicted embodiment of FIG. 1, the circumscribing sidewall 12 extends along the outerwall 18 and presents a sealing surface which extends around the optical socket and nose bridge area and may be include a straight line, arc, polygon or irregular shape for extending along the post-operative surgical area. The elasticity of the elastic band 16 may be adjusted as desired for adjusting the compressive forces exerted by the bilateral compression device 10.

Generally, the central cavity 20 extends interiorly from the outerwall 18 outwardly along the circumscribing sidewall 12 and is adapted for receipt of a plurality of membranes. Alternatively, the central cavity 20 could extend at least partially through the outerwall 18.

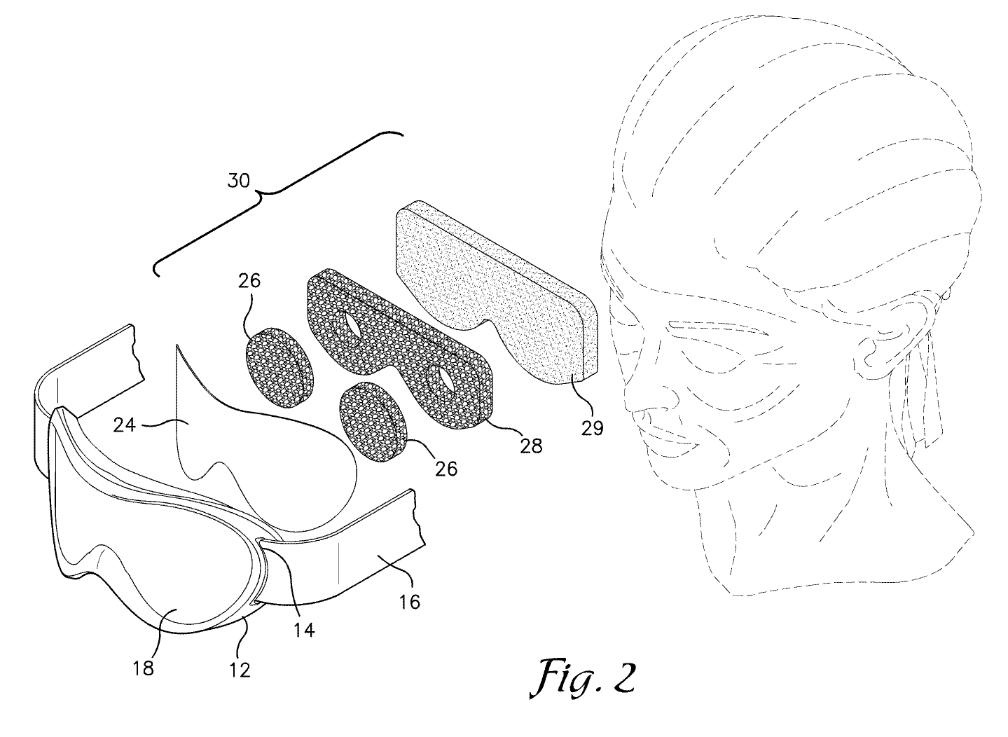

The exemplary embodiment of a post-operative pillow 30 depicted in FIG. 2 includes an outer membrane 24 separated from an inner membrane 29 by a pair of spacers 26 and a compression membrane 28. The spacers 28 are centrally aligned and correspond to the pair of recesses 31 in the compression membrane 28. Generally, the recesses 31 facilitate communication between the spacers 28 and the inner membrane 29 through the compression membrane 28.

Generally, the post-operative pillow 30 is configured for receipt by the central cavity 20. In operation, the post-operative pillow 30 provides multiple compressive forces upon the post-operative surgical area. In the depicted embodiment of FIG. 2, the post-operative pillow 30 provides a reduced compressive force along a central area associated with a pair of recesses 31, a greater compressive force being extended outwardly therefrom towards the facial area surrounding the post-operative surgical area. While the embodiment depicted in FIG. 2 generally provides for a bilateral compression, or two different compression forces, additional compression forces may be utilized in the present invention as desired for selectively reducing the swelling and discomfort associated with the post-operative surgical area to assist in the healing process.

The compression forces exerted upon the post-operative surgical area by the bilateral compression device 10 generally correspond to the properties of the post-operative pillow 30. Generally, two compression forces are provided, a central compression force and a surrounding compression force. The central compression force corresponds to the recess 31, while the surrounding compressive force corresponds to the compression membrane 28. Thus the compression forces exerted upon the post-operative surgical area vary by the configured recess 31 and the selected compression membrane 28. Generally, the desired compression for the central compression force varies in pressure from the surrounding compression force.

The outer membrane 24 is generally an opaque or non-translucent material which is configured for placement between the outerwall 18 and the central cavity 20. The outer membrane 24 may also provide a moisture barrier to prevent any unwanted condensation within the central cavity 20 during use. The outer membrane 24 depicted in FIG. 2, generally has a continuous layer which is adapted for placement on the outerwall 18 along the inner cavity 20 and for receiving the spacers 26. The outer membrane 24 may be secured with adhesive and may have an additional adhesive layer for securing the spacers 26.

The post-operative pillow 30 is configured for receipt within the central cavity 20 and generally provides for compression along the relevant facial contours such as the nose bridge or eye-socket (not shown). In the depicted embodiment of FIG. 2, the post-operative pillow 30 generally includes the facial membrane 29, the compression membrane 28 and the spacer 26. One embodiment of the compression membrane 28 and spacer 26 includes utilization of a generally compressible cellular material which is compressible, moisture resistant and provides thermal insulation. The compressible cellular material associated with the compression membrane 28 and spacers 26 of FIG. 2 may be used either individually or in combination with different or additional material and either configured as a continuous layer or selectively positioned within the central cavity 20 to provide the desired protection and compressibility at the relevant post-operative location. The material used for the spacers 26 and the compression membrane 28 may be the same or they may be different, however, the embodiment depicted in FIG. 2 illustrates them similarly. By way of example, both the compression membrane 28 and the spacers 26 may be, but are not limited to, a laminated cellular foam material which is moisture resistant and has a thickness of around ½″ thick, but at least 1/1641 thick. An example of commercially available material includes ENSOLITE styles IV1, IV2, IV3, IV4, IV5, GIC OR IVC all manufactured by Ensolite, Inc. of Mishawaka, Ind.

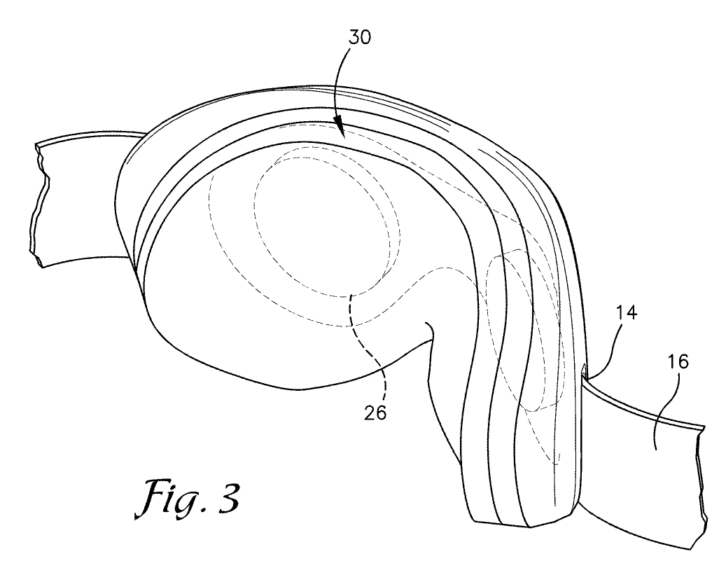

As indicated in FIG. 3, each of the spacers 26 is configured for receipt within the central cavity 20 near the outerwall 18 and placement between the outerwall 18 and the compressible membrane 28. The compressible membrane 28, is configured for placement within the circumscribing sidewall 12 for receipt by the central cavity 20. Generally, the spacers 26 in combination with the compression membrane 28 may be bonded directly to the outerwall 18 to help keep out moisture and maintain the interior temperature within the central cavity 20 during use. Alternatively, in the embodiment depicted in FIG. 2, the spacers 26 may be bonded to the outer membrane 24 and the compression membrane 28 with for example an adhesive material for maintaining the placement of the compression membrane 28 and spacer 26 within the central cavity.

The inner or facial membrane 29 is generally fabricated from a material designed to cushion the treated area of the patient body after the surgical procedure without unnecessarily adhering to the wounded area. During use, the facial membrane 29 may become dirty or soiled from use in connection with the post-operative treatment. Therefore, in one embodiment, the facial membrane 29 may be removed, with a new or fresh facial membrane 29 being applied to the post-operative pillow 30. The facial membrane 29 in the depicted embodiment, is substantially planar with a configuration which generally corresponds to the central cavity 20. The facial membrane 29 may be fabricated from a variety of compressible materials which are suitable for treating wounds but in a preferred situation, it would have a smooth, comfortable surface which would limit irritation or adhesion to the post-operative area. By way of example, and not as a limitation, the inner membrane 29 may be fabricated from a compressible polyurethane foam material having a silicon coating. Examples may include material made by Mentor Corporation under the trade name EPIFOAM or TOPIFOAM. The inner membrane 29 may be secured to the compression membrane 28 or to the circumscribing sidewall 12 mechanically, with for example silk or chemically with for example adhesive.

In one operational embodiment, the googles may be placed on the patient's head while the patient is laying back with the post-operative pillow 30 placed within the central cavity 20, the inner membrane 29 pressed against the facial contours around the post-operative surgical area (not shown). The inner membrane 29 extends across the upper and lower eye (not shown) with the post-operative pillow 30 exerting pressure across each eye region. The pressure may be adjusted with the band 16, with for example a buckle (not shown) or another adjustment mechanism to reduce or increase the pressure as desired. As previously indicated, the circumscribing sidewall 12 presents a sealing surface which extends around the optical socket and nose bridge area associated with the post-operative surgical area (not shown). The googles may be removed as desired and the inner membrane 29 may be removed or changed as needed. Alternatively, the inner membrane 29 may alternatively be configured to warm, cool or to help heal the post-operative surgical area such as by dispensing ointments, drops or other post-operative treatments upon the surrounding post-operative surgical area as desired through for example a use of a medicated dressing which has specially treated membranes which have various known substances impregnated into the material of the inner membrane 29.

It is to be understood that while certain forms of the present invention have been illustrated and described herein, it is not to be limited to the specific forms or arrangement of parts described and shown.

What is claimed and desired to be secured by Letters Patent:

1. A post-operative bilateral compression device comprising:
a central cavity presented by an outerwall and a circumscribing sidewall, said circumscribing sidewall presenting a contoured surface;
a band secured to said circumscribing sidewall for securing said post-operative bilateral compression device during use; and
a post-operative pillow received by said central cavity and further comprising an outer membrane separated by an inner membrane and adapted for exerting a central compressive force and a surrounding compressive force towards a post-operative surgical site.

2. The post-operative bilateral compression device of claim 1 wherein said inner membrane is removable.

3. The post-operative bilateral compression device of claim 1 wherein said inner membrane presents a smooth, non-stick surface.

4. The post-operative bilateral compression device of claim 1 wherein said circumscribing sidewall presents a sealing surface which extends along the post-operative surgical site.

5. The post-operative bilateral compression device of claim 1 further comprising a compression membrane positioned between said inner and outer membrane, said inner membrane in communication with a pair of spacers located between said compression membrane and said outer membrane.

6. The post-operative bilateral compression device of claim 5 wherein said spacers are centrally aligned within said central cavity with a pair of recesses in said compression membrane.

7. The post-operative bilateral compression device of claim 1 wherein said outer membrane limits visibility through said post-operative bilateral compression device.
